(19) United States
(12) Patent Application Publication
(10) Pub. No.: US 2019/0153156 A1
(43) Pub. Date: May 23, 2019

(54) FUNCTIONALIZATION OF TRANS-3-HEXENEDIOIC ACID FOR THE PRODUCTION OF HYDROPHOBIC POLYAMIDES AND CHEMICAL RESISTANCE THEREOF

(71) Applicant: Iowa State University Research Foundation, Inc., Ames, IA (US)

(72) Inventors: JOHN EDWARD MATTHIESEN, Hillsboro, OR (US); JAKE WILLIAM NELSON, Warrenville, IL (US); JEAN-PHILIPPE TESSONNIER, Ames, IA (US); ERIC W. COCHRAN, Ames, IA (US); SANAZ ABDOLMOHAMMADI, Ames, IA (US); NACU HERNANDEZ, Ames, IA (US)

(21) Appl. No.: 16/192,937

(22) Filed: Nov. 16, 2018

Related U.S. Application Data
(60) Provisional application No. 62/750,978, filed on Oct. 26, 2018.
(60) Provisional application No. 62/587,966, filed on Nov. 17, 2017.

(51) Int. Cl. C08G 69/42; C08G 69/26; C08G 69/04

(52) U.S. Cl. CPC C08G 69/42; C07C 323/52; C08G 69/04; C08G 69/265

(57) ABSTRACT

The present invention relates to a polymer comprising a repeating group having the structure of formula (I)

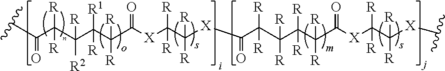

wherein R, R¹, R², X, i, j, m, n, o, and s are as described herein. The present invention also relates to a process for preparation of such polymers.

This application claims the priority benefit of U.S. Provisional Patent Application Ser. No. 62/750,978, filed Oct. 26, 2018, and U.S. Provisional Patent Application Ser. No. 62/587,966, filed Nov. 17, 2017, each of which is hereby incorporated by reference in its entirety.

This invention was made with government support under CBET-1512126 awarded by National Science Foundation and IIP-1701000 awarded by National Science Foundation. The government has certain rights in the invention.

### FIELD OF THE INVENTION

The present invention relates to functionalization of trans-3-hexenedioic acid for the production of hydrophobic polyamides.

### BACKGROUND OF THE INVENTION

Polyamide 6,6 (Nylon 6,6) is a versatile engineering thermoplastic with high strength and stiffness, and thermal stability. These properties make Nylon 6,6 widely applicable in a variety of industries, including automotive parts, electronics, and food packaging. Despite its widespread use, Nylon 6,6 has two major drawbacks: it has a high moisture absorption and limited chemical stability in the presence of aqueous solutions containing high concentrations of inorganic salts. For example, zinc chloride is an aggressive agent that induces cracking among polyamides, making them unsuitable for applications in the automotive industry, in particular for tubing/hosing parts. In this regard, plasticized polyamides, such as Nylon 11 and Nylon 12, with higher ratios of methylene to amide group, show higher resistance to stress cracking compared to Nylon 6,6. However, their lower melting temperature is a barrier to their applications at temperatures above 95° C. Similarly, plasticized Nylon 6,10 and 6,12, present higher melting temperatures compared to Nylon 11 and Nylon 12, but show lower mechanical properties and poor chloride salt resistance, impeding their used for auto tubing applications.

Polyamides are noted for their high strength, toughness, excellent wear properties, and chemical resistance. However, dimensional stability is a major weakness of some nylons, because they easily absorb water, which results in reduction in tensile strength and stiffness while increasing elongation by acting as a plasticizer. For example, the Young's modulus values for Nylon 6,6 and Nylon 6 decrease by about 40% with the absorption of 2% moisture. Nylons with longer monomers are less moisture-sensitive and find use in under-the-hood automotive applications. Conventional polyamides with longer chain are prepared by co-polymerizing hexamethylenediamine (HMDA) with azelaic acid (Nylon 6,9), sebacic acid (Nylon 6,10), and dodecanedioic acid (Nylon 6,12). However, the longer chain has undesirable mechanical properties. For example, Nylon 6,12 has a lower Young's modulus, higher elongation, lower strength, lower thermal distortion temperature, lower hardness, and lower melting point than Nylon 6,6 under dry conditions. Polyamides with longer chains present advantages over Nylon 6,6 only when water uptake is an issue. Therefore, there are unmet needs for moisture insensitive polyamides.

The present invention is directed to overcoming these and other deficiencies in the art.

### SUMMARY OF THE INVENTION

One aspect of the present invention relates to a polymer having the structure of formula (I):

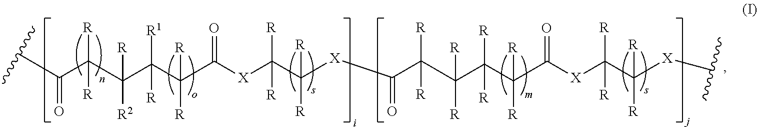

wherein

X is NH or O;

R is independently selected from the group consisting of H and C₁₋₂₀ alkyl;

R¹ and R² are independently selected from the group consisting of H, C₁₋₁₀₀ alkyl, and —S—R³—R⁴;

R³ is C₁₋₁₀₀ alkylene;

R⁴ is selected from the group consisting of —PO₃⁻, —SO₃⁻, —NH₃⁺, —S⁻, —PO₃H, —SO₃H, —NH₂, —SH, and —H;

i is 1 to 1,000,000;

j is 1 to 1,000,000;

m is 1 to 30;

n is 1 to 30;

o is 1 to 30;

s is 1 to 50; and

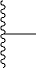

is a terminal group of the polymer;

or a salt thereof.

Another aspect of the present invention relates to a process for preparation of a polymer having the structure of formula (I):

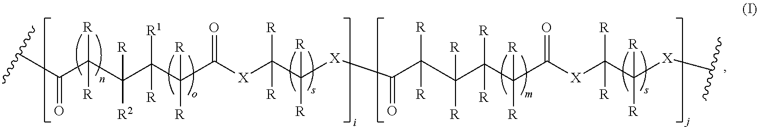

wherein

X is NH or O;

R is independently selected from the group consisting of H and C₁₋₂₀ alkyl;

R¹ and R² are independently selected from the group consisting of H, C₁₋₁₀₀ alkyl, and —S—R³—R⁴;

R³ is C₁₋₁₀₀ alkylene;

R⁴ is selected from the group consisting of —PO₃⁻, —SO₃⁻, —NH₃⁺, —S⁻, —PO₃H, —SO₃H, —NH₂, —SH, and —H;

i is 1 to 1,000,000;

j is 1 to 1,000,000;

m is 1 to 30;

n is 1 to 30;

o is 1 to 30;

s is 1 to 50; and

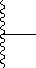

is a terminal group of the polymer;

or a salt thereof.

This process includes:

providing a compound having the structure of formula (II):

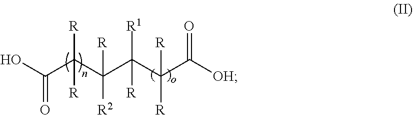

providing a compound having the structure of formula (III):

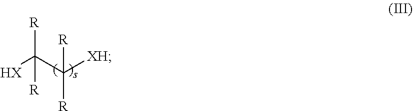

providing a compound having the structure of formula (IV):

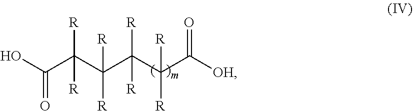

reacting the compound of formula (II), the compound of formula (III), and the compound of formula (IV) under conditions effective to produce the product compound of formula (I).

This invention relates to the synthesis of a hydrophobic and chemical-resistant Bio-Advantaged Nylon (BAN). Reducing water absorption and increasing chemical resistance allows the use of Bio-Advantaged Nylon in new applications were the materials are subject to high humidity and/or to contact with liquid water, and chemical attacks from aqueous halide salts. These materials are for example relevant for use in transportation vehicles as conventional Nylons are sensitive to weather and exposure to salts.

### BRIEF DESCRIPTION OF THE DRAWINGS

FIG. 1 is an image showing hydrophobic Bio-Advantaged Nylon (BAN-H) (functionalized), unsaturated Bio-Advantaged Nylon (BAN) (unfunctionalized), and Nylon 6,6.

FIG. 2 is a ¹H NMR spectrum of purified 3-(dodecylthio)hexanedioic acid (DDTHDA).

FIG. 3 is a ¹H NMR spectrum of purified 3-(hexylthio)hexanedioic acid.

FIGS. 4A-4B are graphs showing differential scanning calorimetry (DSC) plots of comparison of Nylon 6,6 and hydrophobic BAN-H samples from 25-300° C. (FIG. 4A) and thermogravimetric analysis (TGA) plots of comparison of Nylon 6,6 and hydrophobic BAN-H samples from 40-700° C. (FIG. 4B).

FIGS. 5A-5B are graphs showing differential scanning calorimetry (DSC) plots of comparison of Nylon 6,6 and BAN samples from 25-300° C. (FIG. 5A) and thermogravimetric analysis (TGA) plots of comparison of Nylon 6,6 and BAN samples from 40-700° C. (FIG. 5B).

FIG. 6 shows results of the moisture absorption experiments.

FIG. 7 is a graph showing X-ray diffraction (XRD) data of hydrophobic BAN-H.

FIGS. 8A-8B are scanning electron microscopy images showing the surface of conventional Nylon 6,6 (FIG. 8A) and hydrophobic BAN C12-20H containing 10 mol % of DDTHDA (20 mol % relative to adipic acid) after exposure to a zinc chloride solution for 96 hours (FIG. 8B).

### DETAILED DESCRIPTION OF THE INVENTION

One aspect of the present invention relates to a polymer having the structure of formula (I):

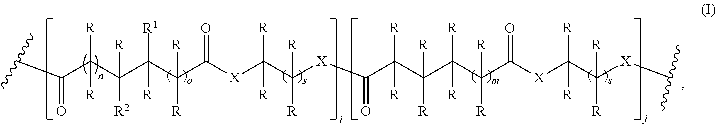

wherein

X is NH or O;

R is independently selected from the group consisting of H and C₁₋₂₀ alkyl;

R¹ and R² are independently selected from the group consisting of H, C₁₋₁₀₀ alkyl, and —S—R³—R⁴;

R³ is C₁₋₁₀₀ alkylene;

R⁴ is selected from the group consisting of —PO₃⁻, —SO₃⁻, —NH₃⁺, —S⁻, —PO₃H, —SO₃H, —NH₂, —SH, and —H;

i is 1 to 1,000,000;

j is 1 to 1,000,000;

m is 1 to 30;

n is 1 to 30;

o is 1 to 30;

s is 1 to 50; and

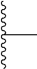

is a terminal group of the polymer;

or a salt thereof.

As used above, and throughout the description herein, the following terms, unless otherwise indicated, shall be understood to have the following meanings. If not defined otherwise herein, all technical and scientific terms used herein have the same meaning as is commonly understood by one of ordinary skill in the art to which this technology belongs. In the event that there is a plurality of definitions for a term herein, those in this section prevail unless stated otherwise.

The term “alkyl” means an aliphatic hydrocarbon group which may be straight or branched having about 1 to about 100 carbon atoms in the chain. Branched means that one or more lower alkyl groups such as methyl, ethyl or propyl are attached to a linear alkyl chain. Exemplary alkyl groups include methyl, ethyl, n-propyl, i-propyl, n-butyl, t-butyl, n-pentyl, and 3-pentyl.

The term “alkylene” refers to a group obtained by removal of a hydrogen atom from an alkyl group. Non-limiting examples of alkylene include methylene and ethylene.

The term “substituted” or “substitution” of an atom means that one or more hydrogen on the designated atom is replaced with a selection from the indicated group, provided that the designated atom's normal valency is not exceeded.

“Unsubstituted” atoms bear all of the hydrogen atoms dictated by their valency. When a substituent is keto (i.e., ═O), then two hydrogens on the atom are replaced. Combinations of substituents and/or variables are permissible only if such combinations result in stable compounds; by “stable compound” or “stable structure” is meant a compound that is sufficiently robust to survive isolation to a useful degree of purity from a reaction mixture.

The term “optionally substituted” is used to indicate that a group may have a substituent at each substitutable atom of the group (including more than one substituent on a single atom), provided that the designated atom's normal valency is not exceeded and the identity of each substituent is independent of the others. Up to three H atoms in each residue are replaced with alkyl, halogen, haloalkyl, hydroxy, lower alkoxy, carboxy, carboalkoxy (also referred to as alkoxycarbonyl), carboxamido (also referred to as alkylaminocarbonyl), cyano, carbonyl, nitro, amino, alkylamino, dialkylamino, mercapto, alkylthio, sulfoxide, sulfone, acylamino, amidino, phenyl, benzyl, heteroaryl, phenoxy, benzyloxy, or heteroaryloxy.

Compounds described herein may contain one or more asymmetric centers and may thus give rise to enantiomers, diastereomers, and other stereoisomeric forms. Each chiral center may be defined, in terms of absolute stereochemistry, as (R)- or (S)-. The present invention is meant to include all such possible isomers, as well as mixtures thereof, including racemic and optically pure forms. Optically active (R)- and (S)-, (−)- and (+)-, or (D)- and (L)-isomers may be prepared using chiral synthons or chiral reagents, or resolved using conventional techniques. When the compounds described herein contain olefinic double bonds or other centers of geometric asymmetry, and unless specified otherwise, it is intended that the compounds include both E and Z geometric isomers. Likewise, all tautomeric forms are also intended to be included.

The term “salts”, when used in relation to the compounds and polymers of the present invention, means the organic acid addition salts and base addition salts of the compounds and polymers of the present invention. Exemplary acid addition salts include the hydrobromide, hydrochloride, sulfate, bisulfate, phosphate, nitrate, acetate, oxalate, valerate, oleate, palmitate, stearate, laurate, borate, benzoate, lactate, phosphate, tosylate, citrate, maleate, fumarate, succinate, tartrate, naphthylate, mesylate, glucoheptonate, lactiobionate, sulphamates, malonates, salicylates, propionates, methylene-bis-b-hydroxynaphthoates, gentisates, isethionates, di-p-toluoyltartrates, methane-sulphonates, ethanesulphonates, benzenesulphonates, p-toluenesulphonates, cyclohexylsulphamates and quinateslaurylsulphonate salts, and the like. Suitable metal salts include the sodium, potassium, calcium, barium, zinc, magnesium, and aluminum salts.

The term “salts”, when used in relation to the chemical properties of the polymers of the present invention, means the inorganic salts. Suitable salts include metal, fluorides, metal chlorides, metal bromides, and metal iodides. Preferred salts include CaCl₂, MgCl₂, ZnCl₂, NaCl, KCl, CaBr₂, MgBr₂, ZnBr₂, NaBr, or KBr.

The term “copolymer” refers to a polymer derived from more than one species of monomer.

The term “alternating copolymer” or “alternating polymer” refers to a copolymer consisting of two or more species of monomeric units that are arranged in an alternating sequence (in which every other building unit is different (-M₁M₂-)ₙ.

The term “random copolymer” or “random polymer” refers to a copolymer in which there is no definite order for the sequence of the different building blocks (-M₁M₂M₁M₁M₂M₁M₂M₂-).

The term “statistical copolymer” or “statistical polymer” refers to a copolymer in which the sequential distribution of the monomeric units obeys known statistical laws.

The term “block copolymer” or “block polymer” refers to a macromolecule consisting of long sequences of different repeat units. Exemplary block polymers include, but are not limited to AₙBₘ, AₙBₘAₘ, AₙBₘCₖ, or AₙBₘCₖAₙ.

The repeating groups in the polymer of formula (I) can be the same or different.

In one embodiment, the polymer is a statistical polymer.

In another embodiment, the polymer is a random polymer.

In another embodiment, the polymer is an alternating polymer.

In yet another embodiment, the polymer is a block polymer.

One embodiment relates to the polymer of the present invention where R¹ or R² are independently selected from H and —S—C₁₂H₂₅.

Another embodiment relates to the polymer of the present invention where R¹ or R² are independently selected from H, —S—C₆H₁₃, and —S—C₁₂H₂₅.

Yet another embodiment relates to the polymer of the present invention where X is NH.

Another embodiment relates to the polymer of the present invention having the structure of formula (Ia):

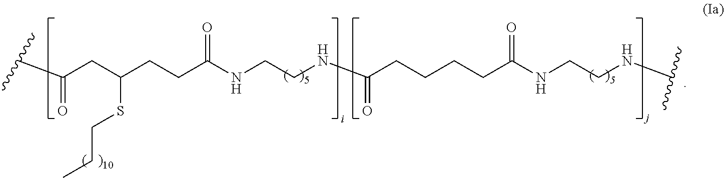

The polymers of the present invention can be prepared according to the schemes described below. Polymers of formula 4 can be prepared by an initial polycondensation reaction (oligomer formation) between acids 1 and 3 and the compound of formula 2 followed by a polymerization step (polymer formation) (Scheme 1). The initial polycondensation reaction (oligomer formation) can be carried out neat or in a variety of solvents, for example in water, methanol (MeOH), ethanol (EtOH), isopropanol (i-PrOH), dimethylformamide (DMF), or other such solvents or in a mixture of such solvents. The initial polycondensation reaction (oligomer formation) can be carried out at a temperature of 0° C. to 150° C., at a temperature of 40° C. to 90° C., or at a temperature of 50° C. to 70° C. The polymer formation step can be performed neat or in a variety of solvents, for example in phenols, cresols, hexafluoro-isopropanol, dimethylformamide (DMF) or other such solvents or in a mixture of such solvents. The final step in the polymerization (polymer formation) reaction can be carried out at a temperature of 20° C. to 400° C., at a temperature of 100° C. to 300° C., or at a temperature of 200° C. to 300° C.

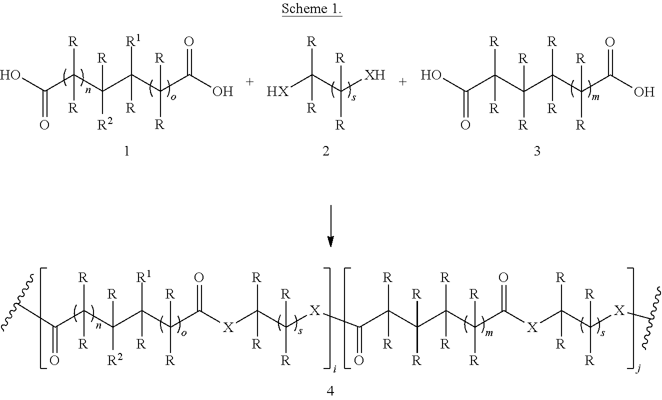

A combination of Nylon 6,6 and DDTHDA is one example of a polymer of formula 4. The combination of Nylon 6,6 and DDTHDA (4a) can be prepared by a polycondensation reaction between 3-(dodecylthio)hexanedioic acid (DDTHDA) (1a), hexamethylenediamine (HMDA) (2a), and adipic acid (3a) (Scheme 2). This reaction can be carried out neat or in a variety of solvents, for example in water, methanol (MeOH), ethanol (EtOH), isopropanol (i-PrOH), dimethylformamide (DMF), phenols, cresols, hexafluoro-isopropanol, tetrafluoroethane (TFE) or other such solvents or in a mixture of such solvents. The reaction can be carried out at a temperature of 0° C. to 400° C., at a temperature 0° C. to 150° C., at a temperature of 40° C. to 90° C., or at a temperature of 50° C. to 70° C., or at a temperature of 100° C. to 300° C., or at a temperature of 200° C. to 300° C.

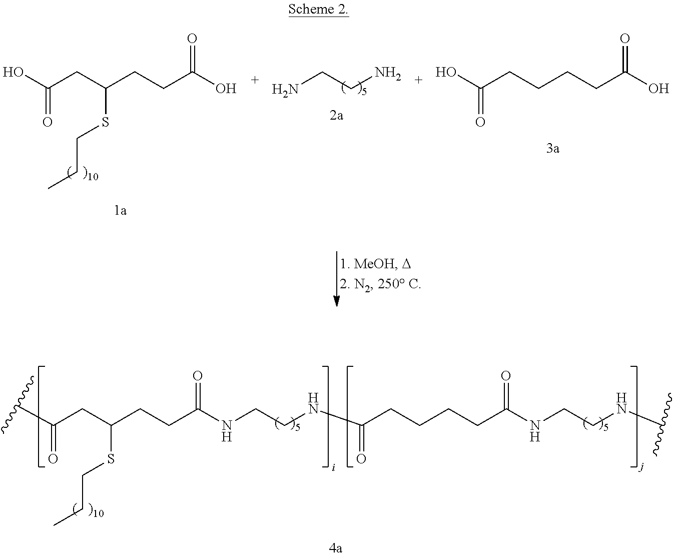

Compounds of formula 1 can be prepared by a reaction between acid 5 and compound of formula 6 (Scheme 3). The reaction can be carried out neat or in a variety of solvents, for example in tetrahydrofuran (THE), methylene chloride (CH₂Cl₂), dimethylformamide (DMF), dioxane or other such solvents or in the mixture of such solvents.

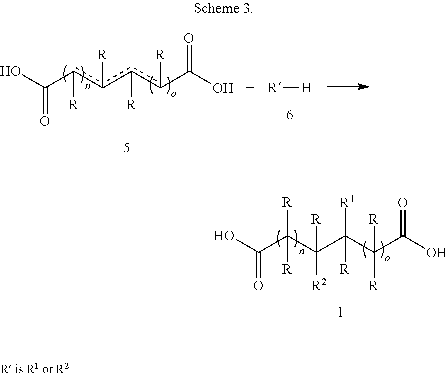

DDTHDA is one of the examples of compounds of formula 1. DDTHDA (1a) can be prepared by reacting 3-hexenedioic acid (5a) and dodecanethiol (6a) (Scheme 4). The reaction can be carried out in THF in the presence of azobisisobutyronitrile (AIBN) or any other suitable azo or peroxide initiators or photoinitiator or a mixture thereof. The reaction can be carried out at room temperature or at the elevated temperature. The reaction can also be carried out under the UV light.

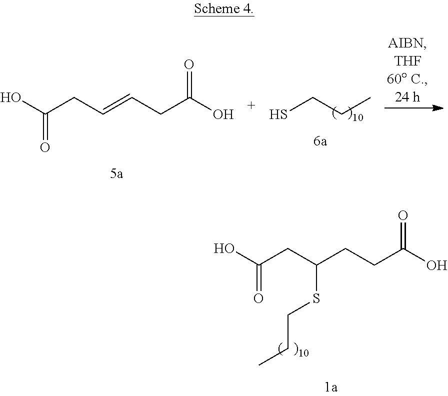

Suitable photoinitiators that can be used in accordance with the present invention include, but are not limited to, benzoin ethers, benzil ketals, α-dialkoxy-acetophenones, α-hydroxy-alkyl-phenones, α-amino-alkyl-phenones, acyl-phosphine oxides, benzo-phenones/amines, thio-xanthones/amines, and titanocenes. Exemplary photoinitiators that can be used include, but are not limited to, acetophenone, anisoin, anthraquinone, anthraquinone-2-sulfonic acid, (benzene)tricarbonyl chromium, benzil, benzoin, benzoin ethyl ether, benzoin isobutyl ether, benzoin methyl ether, benzophenone, benzophenone/1-hydroxycyclohexyl phenyl ketone (50/50 blend), 3,3′,4,4′-benzophenonetetracarboxylic dianhydride, 4-benzoylbiphenyl, 2-benzyl-2-(dimethylamino)-4′-morpholinobutyrophenone, 4,4′-bis(diethylamino)benzophenone, 4,4′-bis(dimethylamino)benzophenone, camphorquinone, 2-chlorothioxanthen-9-one, (cumene)cyclopentadienyliron(ii)hexafluorophosphate, dibenzosuberenone, 2,2-diethoxyacetophenone, 4,4′-dihydroxybenzophenone, 2,2-dimethoxy-2-phenylacetophenone, 4-(dimethylamino)benzophenone, 4,4′-dimethylbenzil, 2,5-dimethylbenzophenone, 3,4-dimethylbenzophenone, diphenyl(2,4,6-trimethylbenzoyl)phosphine oxide/2-hydroxy-2-methylpropiophenone (50/50 blend), 4′-ethoxyacetophenone, 2-ethylanthraquinone, ferrocene, 3′-hydroxyacetophenone, 4′-hydroxyacetophenone, 3-hydroxybenzophenone, 4-hydroxybenzophenone, 1-hydroxycyclohexyl phenyl ketone, 2-hydroxy-2-methylpropiophenone, 2-methylbenzophenone, 3-methylbenzophenone, methybenzoylformate, 2-methyl-4′-(methylthio)-2-morpholinopropiophenone, phenanthrenequinone, 4′-phenoxyacetophenone, thioxanthen-9-one, triarylsulfonium hexafluoroantimonate salts (mixed, 50% in propylene carbonate), triarylsulfonium hexafluorophosphate salts, (mixed, 50% in propylene carbonate).

Another aspect of the present invention relates to a process for preparation of a polymer having the structure of formula (I):

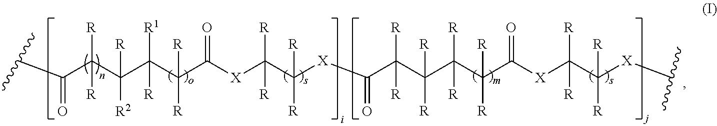

wherein

X is NH or O;

R is independently selected from the group consisting of H and C₁₋₂₀ alkyl;

R¹ and R² are independently selected from the group consisting of H, C₁₋₁₀₀ alkyl, and —S—R³—R⁴;

R³ is C₁₋₁₀₀ alkylene;

R⁴ is selected from the group consisting of —PO₃⁻, —SO₃⁻, —NH₃⁺, —S⁻, —PO₃H, —SO₃H, —NH₂, —SH, and —H;

i is 1 to 1,000,000;

j is 1 to 1,000,000;

m is 1 to 30;

n is 1 to 30;

o is 1 to 30;

s is 1 to 50; and

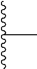

is a terminal group of the polymer;

or a salt thereof.

This process includes:

providing a compound having the structure of formula (II):

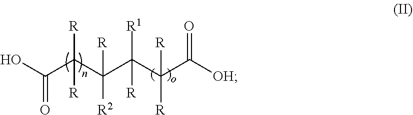

providing a compound having the structure of formula (III):

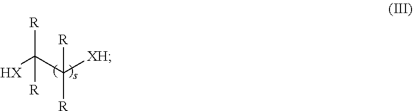

providing a compound having the structure of formula (IV):

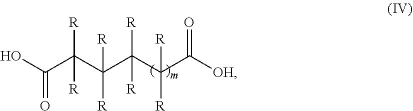

and

reacting the compound of formula (II), the compound of formula (III), and the compound of formula (IV) under conditions effective to produce the product compound of formula (I).

One embodiment relates to the process of the present invention where said providing a compound having the structure of formula (II) includes:

providing a compound having the structure of formula (V):

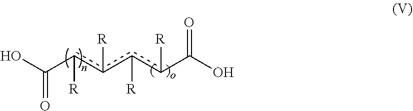

wherein each [symbol] is independently a single or a double bond with no adjacent double bonds, and wherein one [symbol] is a double bond, and

forming the compound having the structure of formula (II) from the compound of formula (V).

According to the present invention, a polymer comprising a repeating group having the structure of formula (I) may include polymers where some of the repeating units have a chemical structure like that of formula (I) but have a double bond resulting from incomplete conversion of the double bond when compound of formula (II) is prepared from the compound of formula (V).

Another embodiment relates to the process of the present invention where the polymer has the structure of formula (Ia):

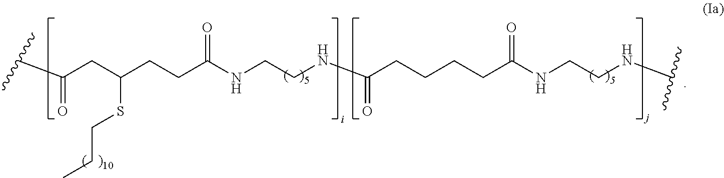

Another embodiment relates to the process of the present invention where the polymer has the structure of formula (Ib):

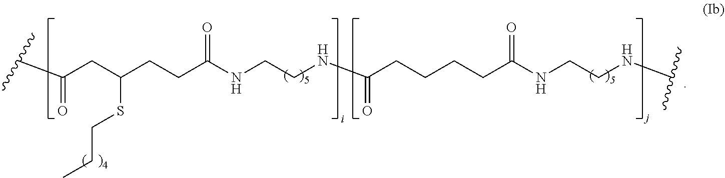

Another embodiment relates to the process of the present invention where the compound of formula (III) is hexamethylenediamine.

Yet another embodiment relates to the process of the present invention where the compound of formula (IV) is adipic acid.

A further embodiment relates to the process of the present invention where the compound of formula (V) is 3-hexenedioic acid.

Yet another embodiment relates to the process of the present invention where said forming the compound having the structure of formula (II) from the compound of formula (V) includes reacting the compound of formula (V) with the compound of formula (VIa):

C₁₋₁₀₀ alkyl-SH (VIa).

Another embodiment relates to the process of the present invention where compound (V) is reacted with the compound of formula (VIa) in the presence of AIBN.

Yet another embodiment relates to the process of the present invention where said forming the compound having the structure of formula (II) from the compound of formula (V) includes reacting the compound of formula (V) with the compound of formula (VIb):

HS—C₁₋₁₀₀ alkyl-S⁻ (VIb).

Another embodiment relates to the process of the present invention where said forming the compound having the structure of formula (II) from the compound of formula (V) includes reacting the compound of formula (V) with the compound of formula (VIc):

HS—C₁₋₁₀₀ alkyl-PO₃⁻ (VIc).

A further embodiment relates to the process of the present invention where said forming the compound having the structure of formula (II) from the compound of formula (V) includes reacting the compound of formula (V) with the compound of formula (VId):

HS—C₁₋₁₀₀ alkyl-SO₃⁻ (VId).

Another embodiment relates to the process of the present invention where said forming the compound having the structure of formula (II) from the compound of formula (V) includes reacting the compound of formula (V) with the compound of formula (VIe):

HS—C₁₋₁₀₀ alkyl-NH₃⁺ (VIe).

Yet another embodiment relates to the process of the present invention where said forming the compound having the structure of formula (II) from the compound of formula (V) includes reacting the compound of formula (V) with the compound of formula (VIb′):

HS—C₁₋₁₀₀ alkyl-SH (VIb′).

Another embodiment relates to the process of the present invention where said forming the compound having the structure of formula (II) from the compound of formula (V) includes reacting the compound of formula (V) with the compound of formula (VIc′):

HS—C₁₋₁₀₀ alkyl-PO₃H (VIc′).

A further embodiment relates to the process of the present invention where said forming the compound having the structure of formula (II) from the compound of formula (V) includes reacting the compound of formula (V) with the compound of formula (VId′):

HS—C₁₋₁₀₀ alkyl-SO₃H (VId′).

Another embodiment relates to the process of the present invention where said forming the compound having the structure of formula (II) from the compound of formula (V) includes reacting the compound of formula (V) with the compound of formula (VIe′):

HS—C₁₋₁₀₀ alkyl-NH₂ (VIe′).

Another embodiment relates to the polymer of Formula (I), having a i:j ratio of from 0.01 to 1. Preferably, the i:j ratio is 0.01, 0.02, 0.03, 0.04, 0.05, 0.06, 0.07, 0.08, 0.09, 0.1, 0.11, 0.12, 0.13, 0.14, 0.15, 0.16, 0.17, 0.18, 0.19, 0.2, 0.21, 0.22, 0.23, 0.24, 0.25, 0.26, 0.27, 0.28, 0.29, 0.3, 0.31, 0.32, 0.33, 0.34, 0.35, 0.36, 0.37, 0.38, 0.39, 0.4, 0.41, 0.42, 0.43, 0.44, 0.45, 0.46, 0.47, 0.48, 0.49, 0.5, 0.6, 0.7, 0.8, 0.9, or 1.0.

The polymer of the present invention may have a molar ratio for the compound having the structure of formula (II):

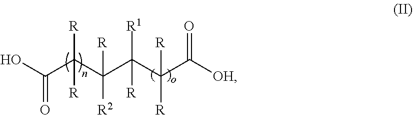

to the compound having the structure of formula (IV):

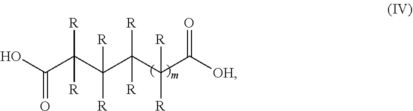

of from 0.01 to 1. Preferably, the molar ratio of the compound of formula (II), to the compound having the structure of formula (IV) is 0.01, 0.02, 0.03, 0.04, 0.05, 0.06, 0.07, 0.08, 0.09, 0.1, 0.11, 0.12, 0.13, 0.14, 0.15, 0.16, 0.17, 0.18, 0.19, 0.2, 0.21, 0.22, 0.23, 0.24, 0.25, 0.26, 0.27, 0.28, 0.29, 0.3, 0.31, 0.32, 0.33, 0.34, 0.35, 0.36, 0.37, 0.38, 0.39, 0.4, 0.41, 0.42, 0.43, 0.44, 0.45, 0.46, 0.47, 0.48, 0.49, 0.5, 0.6, 0.7, 0.8, 0.9, or 1.0.

The polymer of the present invention is desirably able to withstand moisture and aqueous solutions with minimal moisture absorption. In one embodiment, the polymer of the present invention is able to withstand pure water.

The polymer of the present invention is desirably able to withstand moisture and aqueous solutions with less than 10 wt % moisture absorption. Preferably, with less than 1 wt % moisture absorption, less than 0.9 wt %, less than 0.8 wt %, less than 0.7 wt %, less than 0.6 wt %, less than 0.5 wt %, less than 0.4 wt %, less than 0.3 wt %, less than 0.2 wt %, less than 0.1 wt %.

The polymer of the present invention is desirably able to withstand halide solutions for a significant amount of time without any noticeable damage to the polymer. In one embodiment, the polymer is able to withstand halide solutions for 96 hours without any noticeable damage to the polymer. A halide solution according to the present invention is a solution of metal halide, such as solutions of CaCl₂, MgCl₂, ZnCl₂, NaCl, KCl, CaBr₂, MgBr₂, ZnBr₂, NaBr, or KBr. The halide solution can be present at a concentration from 0.1 to 100% (w/v). Preferably, from 1 to 99% (w/v), from 5 to 90% (w/v), from 10 to 80% (w/v), from 20 to 70% (w/v), from 30 to 65% (w/v), from 40 to 60% (w/v), from 45 to 55% (w/v). In one embodiment, the halide solution is a 50% (w/v) solution of CaCl₂, MgCl₂, ZnCl₂, NaCl, KCl, CaBr₂, MgBr₂, ZnBr₂, NaBr, or KBr. In another embodiment, the halide solution is a 50% (w/v) solution of ZnCl₂.

### Examples

### Example 1—Synthesis of 3-(Dodecylthio)hexanedioic Acid (DDTHDA)

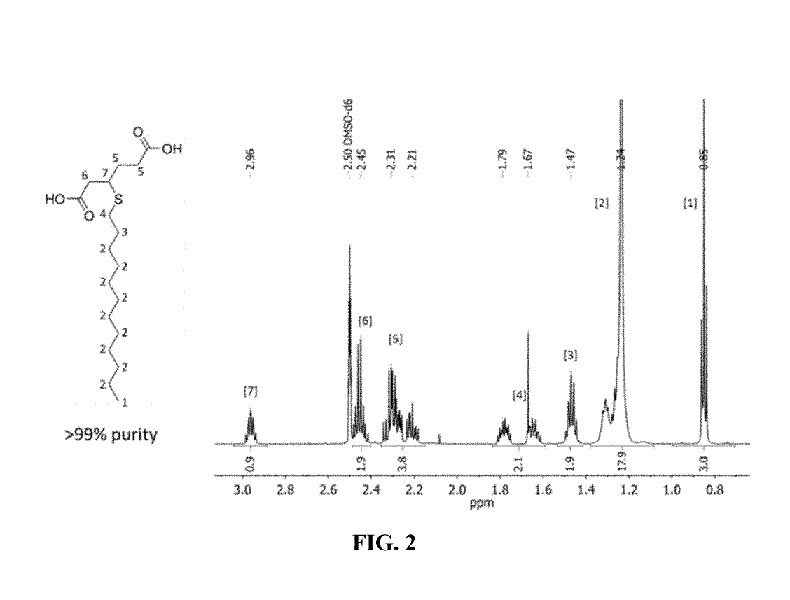

The bioadvantaged trans-3-hexenedioic acid monomer (t3HDA) was functionalized using 1-dodecanethiol (DDT) via thiol-ene chemistry with a thermal based radical initiator such as azobisisobutyronitrile (AIBN). The product of 3-(dodecylthio) hexanedioic acid (DDTHDA) (FIG. 2) was produced using a mixture of t3HDA, AIBN, and DDT in tetrahydrofuran (THF) with the stoichiometric ratio of 1:1:7, respectively. After 72 hours of reaction at 60° C. under inert atmosphere, the THF was evaporated and the product was solubilized in a 1M solution of sodium hydroxide (NaOH). The aqueous solution layer was extracted and then precipitated and purified using 1M hydrochloric acid (HCl). Subsequently, hydrophobic bioadvantaged Nylon (BAN-H) was polymerized using the synthesized DDTHDA, adipic acid (AA), and hexamethylenediamine (HMDA) (following the method described in US Patent Application Publication No. 2017/0130001, which is hereby incorporated by reference in its entirety).

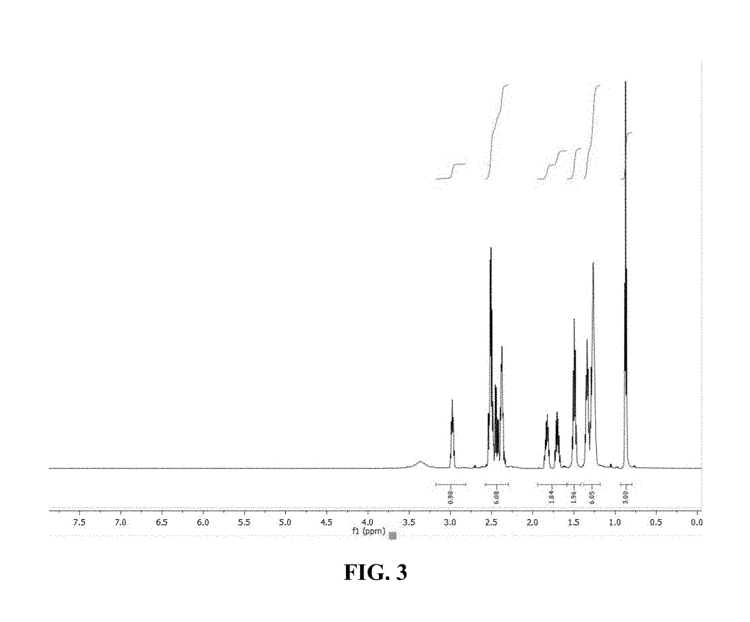

3-(Hexylthio)hexanedioic acid (FIG. 3) can be prepared using similar protocol.

### Example 2—Polymerization of a Hydrophobic and Chemically-Resistant Bio-Advantaged Nylon (BAN)

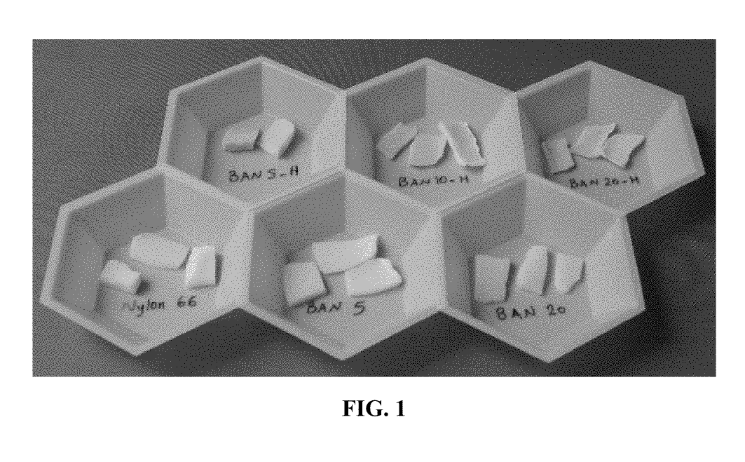

AA and DDTHDA in various molar ratios of 0.05, 0.1 and 0.2 with respect to AA, were both dissolved separately in methanol (CH₃OH), combined, and the resulting solution was mixed in a 1:1 molar ratio with HMDA dissolved in CH₃OH. Then, the reactants were heated in a round bottom flask at 60° C. The precipitated salt was filtered, and left to dry in a fume hood. To complete the polycondensation, the resulting salt was mixed with DI water and heated up to 250° C. under N₂ purge, and then cooled to room temperature. The differences in the color of hydrophobic BAN compared to conventional nylon and unsaturated Nylon are shown in FIG. 1. Samples were named based on the molar ratio of t3HDA and DDTHDA relative to adipic acid with BAN 5 representing an unsaturated Nylon with a 0.05 molar ratio of t3HDA relative to AA, and BAN 5H being a hydrophobic BAN with a 0.05 molar ratio of DDTHDA relative to AA.

### Example 3—Characterization of a Hydrophobic and Chemically—Resistant Bio-Advantaged Nylon (BAN)

Water Absorption Tests

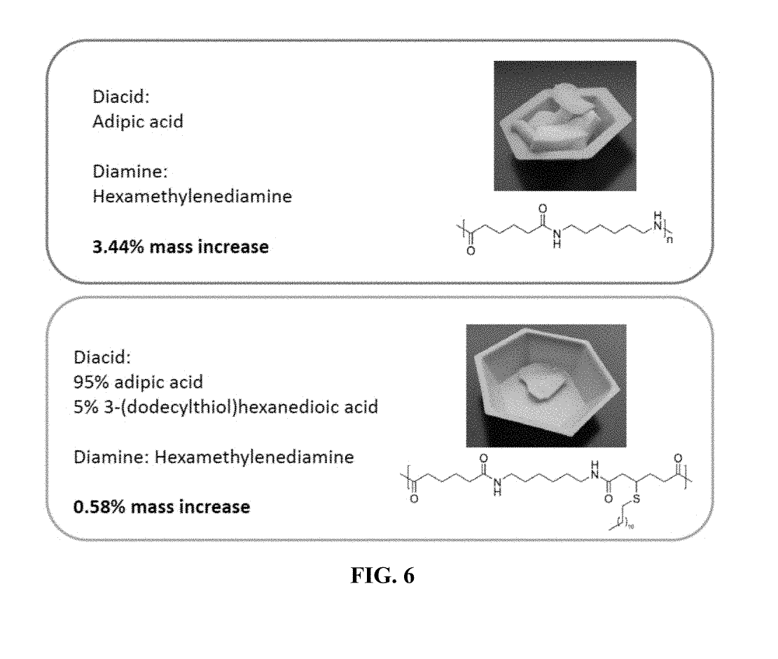

To investigate the effect of the thiol-ene functionalization on the hydrophobicity of the samples, moisture absorption measurements were carried out at room temperature (25° C.) by obtaining a 14×5×1 mm of Nylon 6,6 sample as well as of functionalized BAN samples (FIG. 6). The initial mass of the samples after drying at 60° C. for 48 hours in a vacuum oven were measured using a microbalance (Mettler Toledo model XP2U) with precision of +0.1 mg. The samples were then immersed in 18.2 MΩ DI water for 24 hours. Unabsorbed water was wiped off of the samples' surface after removing them from the water and their masses were quickly measured using the microbalance. Moisture absorption was calculated as follows:

A=[(W−D)/D]×100

where A is moisture absorption, %; W is weight of wet sample, g; D is weight of dry sample, g.

Table 1 shows the moisture absorption results on Nylon samples. The results demonstrate that increasing the amount of DDTHDA into the backbone of the polymer (BAN C12-xH samples, with x representing the percentage of DDTHDA relative to AA) can significantly increase the hydrophobicity of Nylon. The resulting BAN C12-20H reduced moisture absorption (0.28%) compared to the conventional Nylon 6,6 (4.12%).

TABLE 1
Moisture Absorption Comparison for Nylon Samples Containing
Various Amounts of 3HDA Modified with Hexylthiol Groups
(BAN C6-xH with x Corresponding to 5-20 mol % relative to
AA) and Dodecanethiol Groups (BAN C12-xH with x
Corresponding to 5-20 mol % DDTHDA Relative to AA)

| Sample | Moisture Absorption (%) |
|---|---|
| BAN 0 (Nylon6,6) | 4.12 |
| BAN C6-5H | 0.87 |
| BAN C6-10H | 0.40 |
| BAN C6-20H | 0.39 |
| BAN C12-5H | 0.69 |
| BAN C12-10H | 0.51 |
| BAN C12-20H | 0.28 |

Thermal Properties and Crystallinity

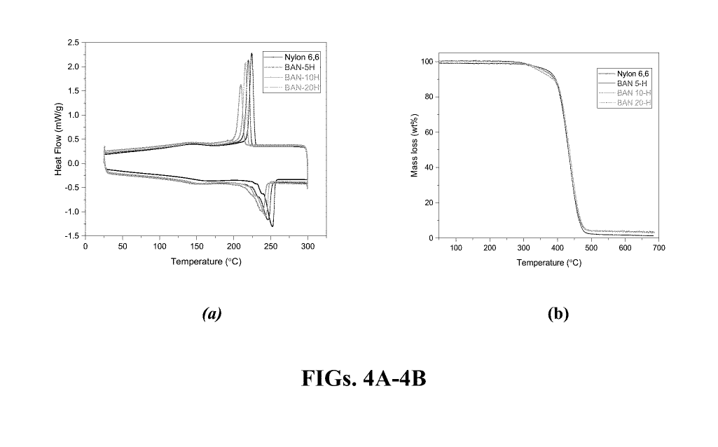

Thermal characterization was performed using differential scanning calorimetry (DSC) and thermogravimetric analysis (TGA). DSC of the polymer powder was performed using a DSC Q2000 (TA Instruments) with aluminum Tzero pans by three consecutive heating and cooling cycles between 25 and 300° C., at a heating-cooling rate of 10° C./min under a 50 mL/min N₂ flow. TGA measurements of all samples were carried out using a NETZSCH model STA 449 F1 Jupiter thermogravimetric analyzer, on 3-5 mg samples placed in alumina crucibles. The samples were heated from room temperature to 700° C. with a heating rate of 10° C./min. Nitrogen with a flow rate of 20 mL/min was used to maintain an inert atmosphere. As an example, the thermal properties of Nylon 6,6 and hydrophobic BAN functionalized with the long chain monothiol (BAN C12) samples are presented in FIGS. 4A-4B. FIG. 4A shows the DSC results for Nylon 6,6 and hydrophobic BAN C12-xH. Nylon 6,6 exhibited a high melting temperature (Tₘ) of 253° C., however, addition of DDTHDA decreased the Tₘ.

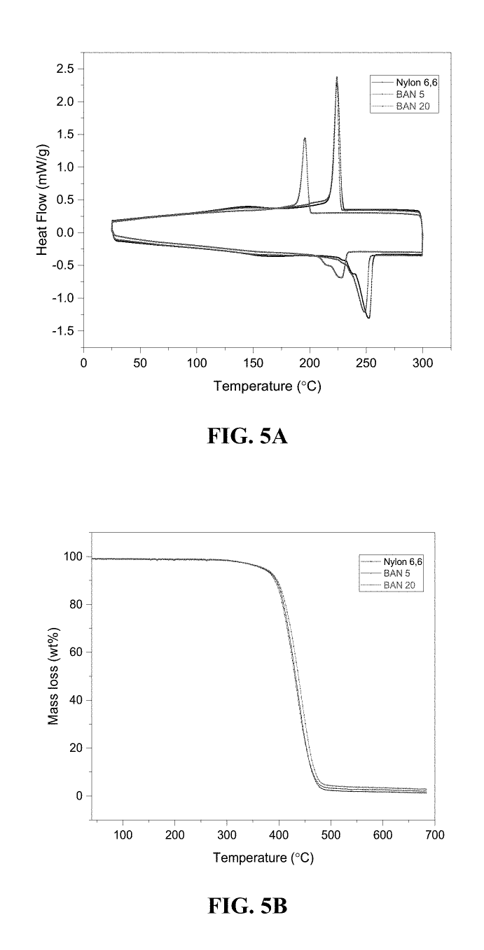

The thermal decomposition of Nylon 6,6 and hydrophobic BAN samples is shown in FIG. 4B. Similar to Nylon 6,6, the hydrophobic bioadvantaged Nylon presented a decomposition temperature ranging between 320 to 500° C. The temperature at 50% weight loss of sample (T_{d50}) varies from 431° C. for Nylon 6,6 and BAN C12-5H to 435° C. for BAN C1210H and BAN C12-20H. The slightly higher value of T_{d50} for hydrophobic BAN samples showed that the addition of long chain thiol has no negative effect on the stability of the polyamide structure. Similar results were obtained for the hydrophobic BAN functionalized with the short chain monothiol (BAN C6) samples (FIGS. 5A-5B).

A Siemens D 500 X-ray diffractometer (XRD) was used to determine the crystallinity of the samples. The diffractometer was equipped with a copper X-ray tube (λ=1.5406 Å) and a diffracted beam monochromator (carbon). Specimens were scanned from 10 to 60 degrees two-theta using a step size of 0.05 degrees and a count time of three seconds per step.

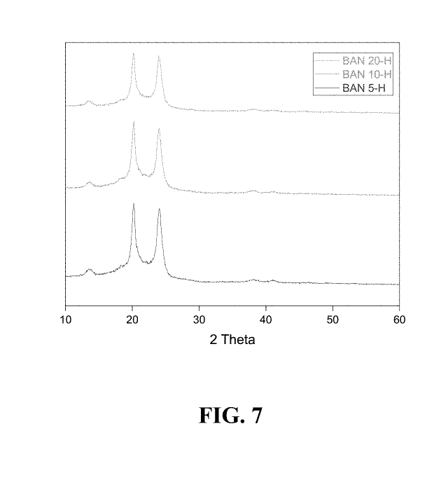

FIG. 7 shows XRD data of hydrophobic BAN samples at room temperature. The diffractogram of hydrophobic BAN is very similar to that of Nylon 6,6 consisting of both an amorphous and a crystalline part. The two characteristic peaks of hydrophobic BAN samples are at the same position as Nylon 6,6 which are approximately at a 20 of 21 and 24 degrees. These peaks correspond to intrasheet and intersheets scattering, respectively, and are characteristic of the α-phase of the triclinic structure. Further addition of functionalized diacid (DDTHDA) slightly changed the crystallinity from 52% for BAN C12-5H to 47% for BAN C12-10H, and 51% for BAN C12-20H. These results are in agreement with the DSC data, and the crystallinity of BAN-20H is consistent with its higher melting point compared to BAN-10H.

Salt Resistance Tests

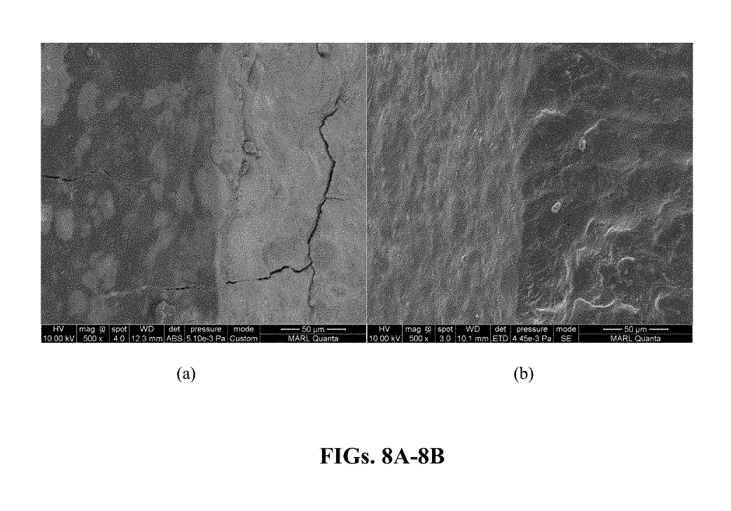

In addition to thermal properties and crystallinity, the hydrophobic BAN samples were tested for their resistance to halides, specifically CaCl₂, MgCl₂, and ZnCl₂. A method commonly used in industry was followed to test the samples. In this regard, samples were submerged into 50% (w/v) of each halide solution for 96 hours at room temperature, recovered, and then dried at 40° C., followed by characterization using scanning electron microscopy (SEM). FIGS. 8A-8B show the SEM results of the Nylon 6,6 and hydrophobic BAN specifically BAN C12-20H. FIG. 8A showed large cracks that extended over several tens of microns on Nylon 6,6. However, FIG. 8B showed Hydrophobic BAN C12-20H has excellent resistance to zinc chloride cracking and it can withstand a 50% (w/v) ZnCl₂ solution for 96 hours without any noticeable damage to the polymer material.

Although preferred embodiments have been depicted and described in detail herein, it will be apparent to those skilled in the relevant art that various modifications, additions, substitutions, and the like can be made without departing from the spirit of the invention and these are therefore considered to be within the scope of the invention as defined in the claims which follow.

What is claimed:

1. A polymer having the structure of formula (I): 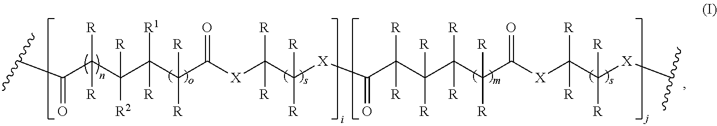
wherein
X is NH or O;
R is independently selected from the group consisting of H and C₁₋₂₀ alkyl;
R¹ and R² are independently selected from the group consisting of H, C₁₋₁₀₀ alkyl, and —S—R³—R⁴;
R³ is C₁₋₁₀₀ alkylene;
R⁴ is selected from the group consisting of —PO₃⁻, —SO₃⁻, —NH₃⁺, —S⁻, —PO₃H, —SO₃H, —NH₂, —SH, and —H;
i is 1 to 1,000,000;
j is 1 to 1,000,000;
m is 1 to 30;
n is 1 to 30;
o is 1 to 30;
s is 1 to 50; and 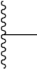
is a terminal group of the polymer;
or a salt thereof.

2. The polymer of claim 1, wherein the polymer is a statistical polymer.

3. The polymer of claim 1, wherein the polymer is a random polymer.

4. The polymer of claim 1, wherein the polymer is an alternating polymer.

5. The polymer of claim 1, wherein the polymer is a block polymer.

6. The polymer according to claim 1, wherein R¹ or R² are independently selected from the group consisting of H and —S—C₁₂H₂₅.

7. The polymer according to claim 1, wherein R¹ or R² are independently selected from the group consisting of, —S—C₁₂H₂₅, and —S—C₆H₁₃.

8. The polymer according to claim 1, wherein X is NH.

9. The polymer according to claim 1, wherein the polymer has the structure of formula (Ia):

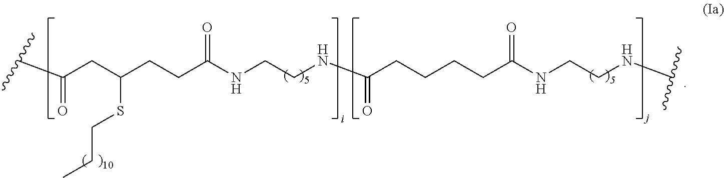

10. The polymer according to claim 1, wherein the polymer has the structure of formula (Ib):

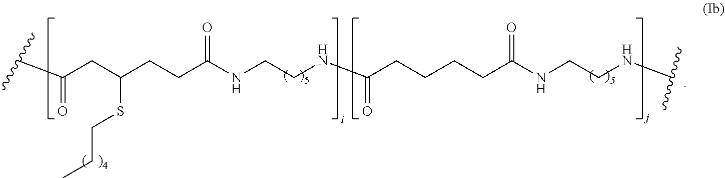

11. A process for preparation of a polymer having the structure of formula (I): 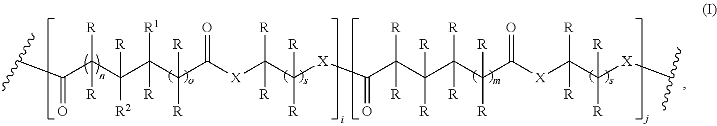
wherein
X is NH or O;
R is independently selected from the group consisting of H and C₁₋₂₀ alkyl;
R¹ and R² are independently selected from the group consisting of H, C₁₋₁₀₀ alkyl, and —S—R³—R⁴;
R³ is C₁₋₁₀₀ alkylene;
R⁴ is selected from the group consisting of —PO₃⁻, —SO₃⁻, —NH₃⁺, —S⁻, —PO₃H, —SO₃H, —NH₂, —SH, and —H;
i is 1 to 1,000,000;
j is 1 to 1,000,000;
m is 1 to 30;
n is 1 to 30;
o is 1 to 30;
s is 1 to 50; and 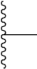
is a terminal group of the polymer;
or a salt thereof,
said process comprising:
providing a compound having the structure of formula (II): 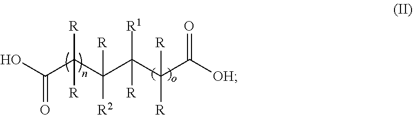
providing a compound having the structure of formula (III): 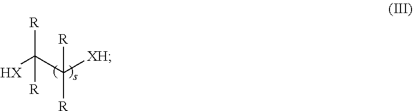
providing a compound having the structure of formula (IV):
and 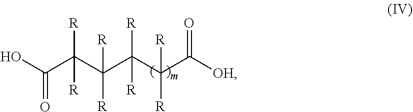
reacting the compound of formula (II), the compound of formula (III), and the compound of formula (IV) under conditions effective to produce the polymer of formula (I).

12. The process according to claim 11, wherein the polymer is a statistical polymer.

13. The process according to claim 11, wherein the polymer is a random polymer.

14. The process according to claim 11, wherein the polymer is an alternating polymer.

15. The process according to claim 11, wherein the polymer is a block polymer.

16. The process according to claim 11, wherein said providing a compound having the structure of formula (II) comprises:
providing a compound having the structure of formula (V): 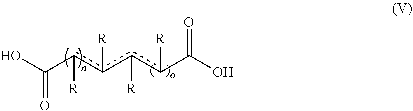
wherein each [symbol] is independently a single or a double bond with no adjacent double bonds, and wherein one [symbol] is a double bond, and
forming the compound having the structure of formula (II) from the compound of formula (V).

17. The process according to claim 11, wherein R¹ or R² are independently selected from the group consisting of H and —S—C₁₂H₂₅.

18. The process according to claim 11, wherein R¹ or R² are independently selected from the group consisting of H, —S—C₆H₁₃, and —S—C₁₂H₂₅.

19. The process according to claim 11, wherein X is NH.

20. The process according to claim 11, wherein the polymer has the structure of formula (Ia):

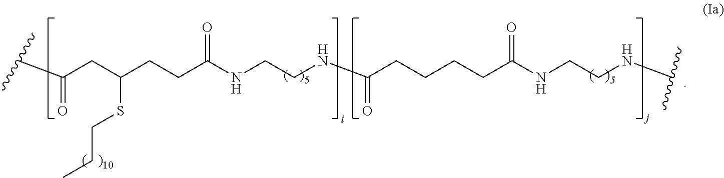

21. The process according to claim 11, wherein the polymer has the structure of formula (Ib):

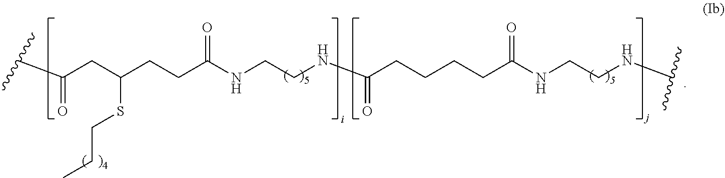

22. The process according to claim 11, wherein the compound of formula (III) is hexamethylenediamine.

23. The process according to claim 11, wherein the compound of formula (IV) is adipic acid.

24. The process according to claim 16, wherein the compound of formula (V) is 3-hexenedioic acid.

25. The process according to claim 16, wherein said forming the compound having the structure of formula (II) from the compound of formula (V) comprises:
reacting the compound of formula (V) with the compound of formula (VIa):
C₁₋₁₀₀ alkyl-SH (VIa).

26. The process according to claim 16, wherein said forming the compound having the structure of formula (II) from the compound of formula (V) comprises:
reacting the compound of formula (V) with the compound of formula (VIb):
HS—C₁₋₁₀₀ alkyl-SH (VIb).

27. The process according to claim 16, wherein said forming the compound having the structure of formula (II) from the compound of formula (V) comprises:
reacting the compound of formula (V) with the compound of formula (VIc):
HS—C₁₋₁₀₀ alkyl-PO₃⁻ (VIc).

28. The process according to claim 16, wherein said forming the compound having the structure of formula (II) from the compound of formula (V) comprises:
reacting the compound of formula (V) with the compound of formula (VId):
HS—C₁₋₁₀₀ alkyl-SO₃⁻ (VId).

29. The process according to claim 16, wherein said forming the compound having the structure of formula (II) from the compound of formula (V) comprises:
reacting the compound of formula (V) with the compound of formula (VIe):
HS—C₁₋₁₀₀ alkyl-NH₃⁺ (VIe).

30. The process according to claim 25, wherein said reacting the compound of formula (V) with the compound of formula (VIa) is carried out in the presence of AIBN.

31. The polymer according to claim 1, wherein the polymer has a i:j ratio of from 0.01 to 1.

32. The process according to claim 11, wherein the polymer has a molar ratio of the compound having the structure of formula (II): 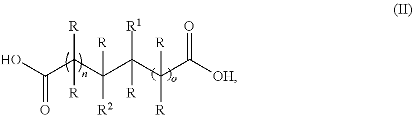
to the compound having the structure of formula (IV): 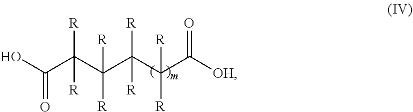
is from 0.01 to 1.

33. The polymer according to claim 1, where the polymer is able to withstand moisture and aqueous solutions with minimal moisture absorption.

34. The polymer according to claim 33, where the polymer is able to withstand pure water with minimal moisture absorption.

35. The polymer according to claim 33, where the polymer is able to withstand moisture and aqueous solutions with less than 1 wt % moisture absorption.

36. The polymer according to claim 35, where the polymer is able to withstand moisture and aqueous solutions with less than 0.5 wt % moisture absorption.

37. The polymer according to claim 33, where the polymer is able to withstand halide solutions for at least 96 hours without any noticeable damage.

38. The polymer according to claim 37, wherein the halide solution is a solution of metal halide selected from the group consisting of CaCl₂, MgCl₂, ZnCl₂, NaCl, KCl, CaBr₂, MgBr₂, ZnBr₂, NaBr, and KBr.
